(19) United States
(12) Patent Application Publication
(10) Pub. No.: US 2026/0046589 A1
(43) Pub. Date: Feb. 12, 2026

(54) Location Tracking and Safety Wearable Device and System

(71) Applicants: Kiera Kampe, Marietta, GA (US); Lashawndra Latham, Marietta, GA (US)

(72) Inventors: Kiera Kampe, Marietta, GA (US); Lashawndra Latham, Marietta, GA (US)

(21) Appl. No.: 19/014,315

(22) Filed: Jan. 9, 2025

Related U.S. Application Data
(60) Provisional application No. 63/679,661, filed on Aug. 6, 2024.

(51) Int. Cl. H04W 4/021; A61B 5/00; A61B 5/024; A61B 5/11

(52) U.S. Cl. CPC H04W 4/021; A61B 5/0004; A61B 5/02438; A61B 5/1112; A61B 5/681; A61B 5/742; A61B 5/746; A61B 5/748

(57) ABSTRACT

The present invention is a safety and tracking system comprising a wearable device and a software application for real-time monitoring and communication. The wearable device includes an adjustable strap with a secure locking mechanism and an embedded control unit. The control unit features a GPS module for accurate location tracking, a push button for sending SOS alerts, and a wireless communication module for data exchange with a remote device. The device is further equipped with a proximity range detector to monitor geofence boundaries, a vital sensor for detecting physiological data and distress events, and a touch display for two-way communication. The software application is installed on a remote electronic device and provides caregivers with real-time location tracking on a digital map, alert notifications, geofence management, and access to historical movement and physiological data of the wearer.

### CROSS-REFERENCE TO RELATED APPLICATION

The present application claims priority to, and the benefit of, U.S. Provisional Application No. 63/679,661 which was filed on Aug. 6, 2024, and is incorporated herein by reference in its entirety.

### FIELD OF THE INVENTION

The present invention generally relates to wearable safety and tracking devices. More specifically, the invention pertains to a wearable device designed to enhance the safety and monitoring of individuals by incorporating real-time GPS tracking, geofencing, and emergency communication capabilities. The safety and tracking wearable device includes a durable, water-resistant, shock-resistant, and break-resistant wristband with an adjustable strap and secure locking mechanism to ensure continuous use. The device features an embedded control unit containing a GPS module for accurate location tracking, a push button for sending SOS alerts, and a vital sensor for detecting heart rate and falls. The device communicates with a remote caregiver's smartphone via a wireless module. Accordingly, the present disclosure makes specific reference thereto. Nonetheless, it is to be appreciated that aspects of the present invention are also equally applicable to other like applications, devices, and methods of manufacture.

### BACKGROUND

By way of background, individuals, particularly children, the elderly, and those requiring medical or specialized care require constant tracking for their safety. Traditional communication devices such as smartphones are used for tracking where calls are used for tracking. However, traditional devices often suffer from significant limitations. Such devices can be easily lost, broken, or stolen, rendering them ineffective in critical moments when they are needed the most. Individuals who go missing, become injured, or are abducted are frequently left without a means to communicate their location or call for help. The lack of a reliable defense mechanism leaves them vulnerable to harm, especially in time-sensitive emergencies.

Patients, such as those with cognitive impairments or medical conditions, who wander away from care facilities are exposed to immediate risks, including accidents or exposure to unsafe environments. In many cases, guardians and emergency responders are only made aware of the situation long after it has occurred, drastically reducing the chances of a timely and effective intervention. Individuals desire a reliable and durable safety device which enables proactive responses to emergencies and enhances the overall safety of vulnerable individuals.

Therefore, there exists a long-felt need in the art for a wearable safety and tracking device that provides durable and reliable real-time location tracking for vulnerable individuals such as children, elderly individuals, and patients requiring care. There is a long-felt need for a wearable device that remains securely attached to the user to prevent accidental removal or tampering, thereby ensuring continuous operation. Additionally, there is a long-felt need for a solution that can discreetly alert caregivers or emergency responders in case of distress or emergencies. Further, there is a long-felt need for a device that can withstand everyday wear and tear, including exposure to water, shocks, and impacts, while providing critical safety and tracking functions. Finally, there is a long-felt need for a device that incorporates geofencing technology to notify caregivers when the user exits (i.e., outside of) preconfigured safe zones, enabling proactive and timely intervention.

The subject matter disclosed and claimed herein, in one embodiment, comprises a wearable safety and tracking device designed for real-time monitoring and communication. The device includes a water-resistant, shock-resistant, and break-resistant wristband with an adjustable strap and secure locking mechanism. The embedded control unit within the wristband features an internal GPS module for continuous location tracking, a push button for activating SOS alerts, and a proximity range detector for monitoring geofence boundaries. The device also incorporates a vital sensor for detecting physiological data such as heart rate and falls, automatically (i.e., autonomously) sending notifications to caregivers when distress is detected. A wireless communication module enables the device to exchange data with a paired remote device, such as a smartphone, via Bluetooth or cellular networks. The top surface of the control unit includes a touch display for displaying alerts, messages, and notifications and enables two-way communication between the wearer and caregiver.

In this manner, the device of the present invention accomplishes all of the foregoing objectives and provides a user-friendly, durable, and secure solution for enhancing the safety of individuals. The wristband remains securely fastened to the user, preventing tampering or accidental removal, and ensures continuous tracking. The integrated geofencing and SOS alert features enable caregivers to respond proactively to emergencies or boundary breaches. Additionally, the device has robust construction and long-lasting durability in various environments. Further, the inclusion of a touch display and two-way communication capabilities eliminates the need for additional devices, enabling effective communication between the wearer and caregiver.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a safety and tracking wearable device. The device comprises a strap configured to be worn around a user's wrist, an embedded control unit is disposed at the center of the strap, wherein the control unit comprises an internal GPS module configured to continuously and accurately track the location of the wearer, a push button configured to send an SOS signal or emergency alert to a remote device when pressed and held for a predetermined duration, a wireless communication module configured to establish a communication channel with one or more paired remote devices, a rechargeable and replaceable battery, a proximity range detector, and a vital sensor configured to monitor heart rate and detect falls.

In another aspect, the device includes a touch display configured to display alerts, text messages, or notifications received from a remote device and facilitate text communication between the wearer and the remote device.

In another embodiment, a wearable safety and tracking device is described. The device includes a strap with a locking mechanism, wherein the locking mechanism includes a continuous loop therein, one or more locking pins, and apertures on the strap. The locking pins are configured to engage with the apertures to securely fasten the device. A control unit is embedded in the strap and includes an internal GPS module, a push button configured to send an emergency alert to a paired remote device, a wireless communication module configured to facilitate exchange of data between the wearable device and the remote device, wherein the locking mechanism is unfastened only when a digital guardian key is transmitted to the control unit or after a preset timer expires.

In one embodiment, a safety and tracking system is disclosed. The system includes a wearable device, a software application installed on a remote electronic device, wherein the software application is configured to receive real-time location data and alerts from the wearable device, display the wearer's location on a digital map, configure geofence boundaries for the wearable device, and enable two-way communication between the caregiver and the wearer via the wearable device.

In yet another aspect, a method for ensuring safety and tracking a wearer is described. The method includes the steps of receiving, by a software application on a remote electronic device, real-time location data from a wearable safety device via a wireless communication channel, displaying, on a digital map in the software application, the current location of the wearer, sending an alert to the remote electronic device when the wearable safety device exits (i.e., outside of) a preset geofence area, detecting, by a vital sensor in the wearable device, physiological data including heart rate and falls, transmitting an automatic distress notification from the wearable device to the remote electronic device when no physiological data is detected for a preconfigured time period, and enabling two-way text communication between the wearable device and the remote electronic device via a touch display on the wearable device.

Numerous benefits and advantages of this invention will become apparent to those skilled in the art to which it pertains upon reading and understanding of the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of safety and tracking wearable device of the present invention in accordance with the disclosed structure;

FIG. 2 illustrates a schematic view showing communication between the safety and tracking wearable device and a remote device in accordance with one embodiment of the present invention;

FIG. 3 illustrates an enlarged view of the fastening mechanism of the safety and tracking device of the present invention in accordance with one embodiment of the present invention;

FIG. 4 illustrates an exemplary user interface displayed by the software application for displaying location of the wearer of the safety and tracking device in accordance with the disclosed architecture; and

FIG. 5 illustrates a text message being displayed on the touch screen of the safety and tracking device of the present invention in accordance with the disclosed structure.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there exists a long-felt need in the art for a wearable safety and tracking device that provides durable and reliable real-time location tracking for vulnerable individuals such as children, elderly individuals, and patients requiring care. There is a long-felt need for a wearable device that remains securely attached to the user to prevent accidental removal or tampering, thereby ensuring continuous operation. Additionally, there is a long-felt need for a solution that can discreetly alert caregivers or emergency responders in case of distress or emergencies. Further, there is a long-felt need for a device that can withstand everyday wear and tear, including exposure to water, shocks, and impacts, while providing critical safety and tracking functions. Finally, there is a long-felt need for a device that incorporates geofencing technology to notify caregivers when the user exits (i.e., outside of) preconfigured safe zones, enabling proactive and timely intervention.

The present invention, in one exemplary embodiment, is a safety and tracking system. The system includes a wearable device, a software application installed on a remote electronic device, wherein the software application is configured to receive real-time location data and alerts from the wearable device, display the wearer's location on a digital map, configure geofence boundaries for the wearable device, and enable two-way communication between the caregiver and the wearer via the wearable device.

Reference will now be made in detail to the present preferred embodiments of the invention, examples of which are illustrated in the accompanying drawings. Wherever possible, the same reference numerals are used in the drawings and the description to refer to the same or like parts.

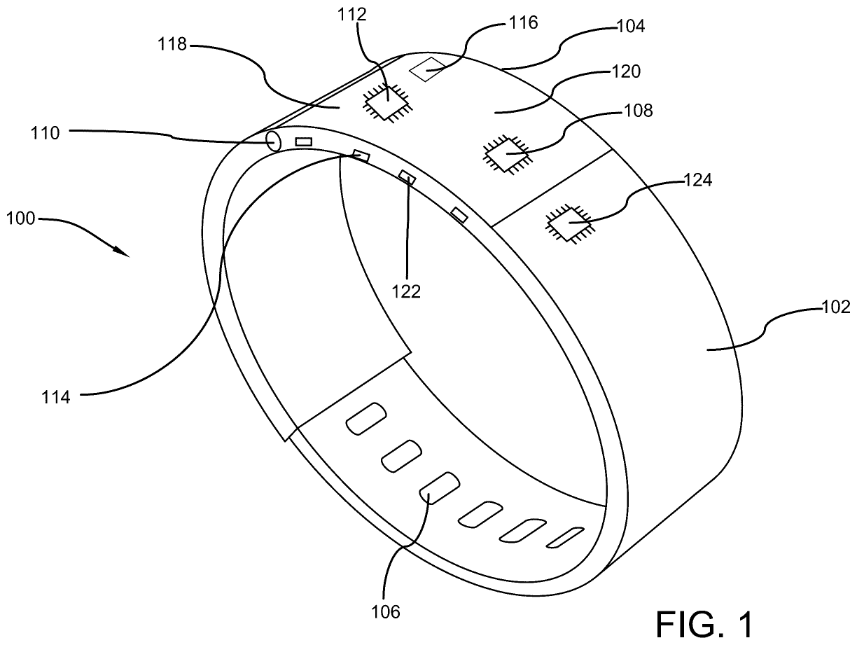

Referring initially to the drawings, FIG. 1 illustrates a perspective view of safety and tracking wearable device of the present invention in accordance with the disclosed structure. The safety and tracking wearable device 100 of the present invention is designed as a wearable device 100 preferably in the form of a smart band, bracelet or any other wearable device. The wearable device 100 is preferably designed to be worn around the wrist (i.e., wristband) of a user and is constructed from water-resistant, shock-resistant, and break-resistant materials. More specifically, the device 100 includes a removably attached strap 102 with an embedded control sensor unit 104. The strap 102 includes a plurality of adjustment apertures 106 disposed therein for adjusting size of the strap 102 to fit users of different sizes.

The embedded control unit 104 is disposed preferably at the center of the strap 102, thereby enabling the control unit 104 to adhere to skin of the wearer wearing the device 100. The control unit 104 includes an internal GPS module 108 which is configured to track the wearer's location. The GPS module 108 is low powered and uses conventional GPS technology to accurately and continuously track location of the wearer of the device 100. An emergency alert button 110 is disposed on the control unit 104 and is configured to send an SOS signal or emergency alert when pushed by the wearer of the device 100. The emergency alert button 110 helps the wearer to send an immediate notification to a caregiver or parent (i.e., paired remote device) wherein the notification includes location of the wearer and a preconfigured alert message. In the preferred embodiment, the wearer presses and holds the alert button 110 for a predetermined duration such as 6 seconds to activate (i.e., actuate) the emergency alert.

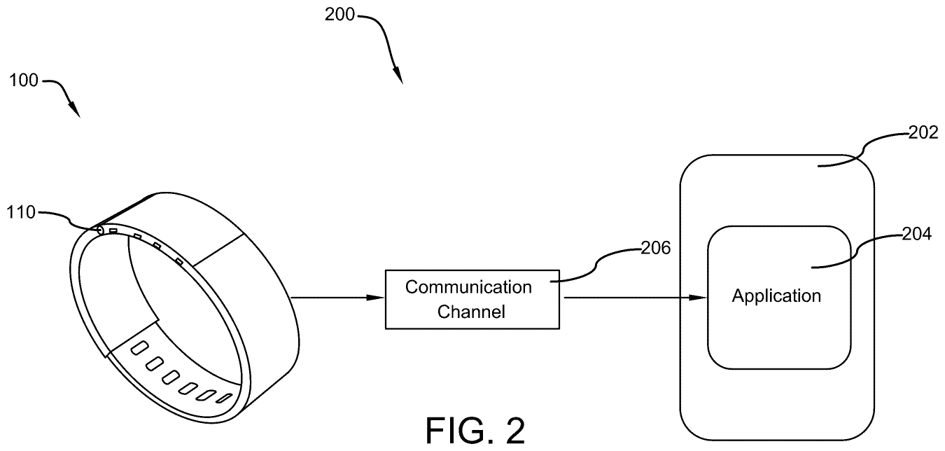

A wireless module 112 establishes a wireless communication channel with one or more paired remote devices for facilitating exchange of SOS signal or emergency alert and other information from the device 100 to the remote device (as illustrated in FIG. 2). The wireless module 112 can establish low energy Bluetooth channel, any 802.11 channel and in some embodiments, can establish IoT communication channel. For providing electric power to the control unit 104, rechargeable and replaceable battery 114 is disposed in the control unit 104. The battery 114 is preferably a button battery but any conventional battery can be used in the device 100.

A vital sensor 116 is included in the device 100 and is configured to detect heart rate and fall detection. The vital sensor 116 is used by the device 100 to detect if the wearer is potentially in distress and the control unit 104 can automatically (i.e., autonomously) send a notification to a caretaker or parent (i.e., paired remote device) when the vital sensor 116 does not detect any information for a predetermined time period such as 1 minute, 30 seconds, or any other preconfigured time duration.

The top surface 118 of the control unit 104 is a touch display and is configured to display an alert, text message or the like received from the remote device. The touch display 120 enables the wearer to communicate and send text messages to the caregiver without using any additional communication device. The device 100 includes a plurality of LEDs 122 which are configured to illuminate when an alert or message is received by the device 100 or when the silent emergency alert button 110 is pushed by the wearer of the device. The LEDs 122 provide a visual alert to the wearer for the functionality of the device 100.

A proximity range detector 124 disposed in the device 100 monitors location of the wearer and sends an alert if the device exits (i.e., outside of) a preset geofence area wherein the area is preconfigured using the application.

FIG. 2 illustrates a schematic view showing communication between the safety and tracking wearable device and a remote device in accordance with one embodiment of the present invention. The safety and tracking device 100 is adapted to communicate with a remote electronic device computing device 202 which includes a software application 204 installed therein. The remote device 202 can be a smartphone or any other compatible device such as a tablet or computer. The remote device 202 is preferably used by a caregiver or a parent and the application 204 enables the caregiver to access real-time location data and alerts from the safety device 100.

The wristband 100 communicates with the application 204 via a communication channel 206 such as Bluetooth (when the devices 100, 202 are nearby) or cellular networks (for long-distance monitoring of the device 100). The application 204 is also configured to remotely activate and deactivate the device 100 while enabling additional configuration parameters such as setting a geofence for the safety device 100. The software application 204 is a specially designed application for interpreting and processing signals received from the safety device 100 and may incorporate machine learning for predictive analytics for recognizing unusual movement patterns of the wearer of the safety device 100 that may indicate distress.

The safety wristband 100 and the application 204 together form the safety and tracking system 200 which enables parents to monitor children, ensuring their safety during outdoor activities, school commutes, or vacations. The system 200 also helps caregivers keep track of elderly individuals with conditions like dementia, reducing the risk of wandering or getting lost. The silent emergency alert button 110 enables the wearer to discreetly call for help during emergencies without drawing attention.

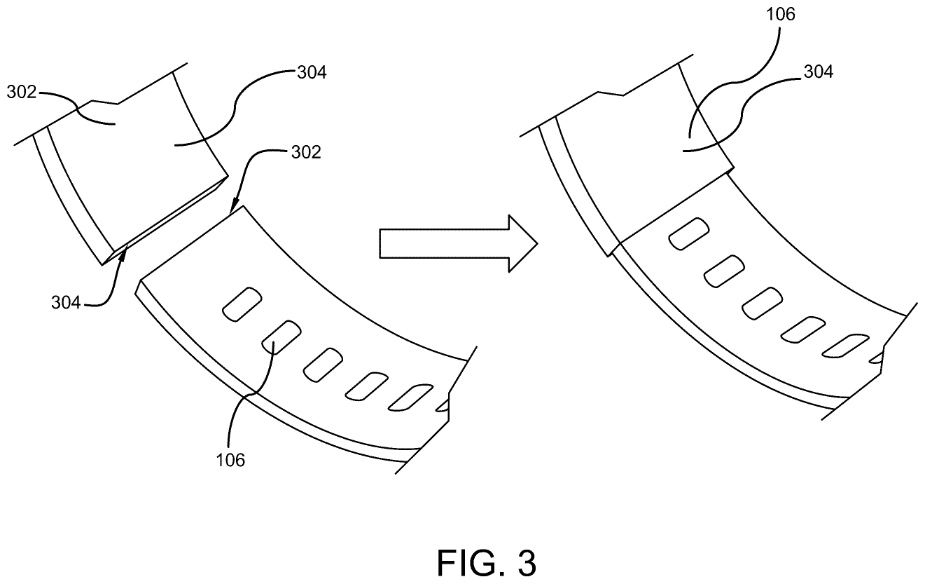

FIG. 3 illustrates an enlarged view of the fastening mechanism of the safety and tracking device of the present invention in accordance with one embodiment of the present invention. The device 100 is securely fastened to the wearer for preventing accidental removal or tampering. In the preferred embodiment, a guardian key which is a digital key and is configured using the application 204 is used for unlocking the device 100. For interlocking the device 100, first end 302 of the strap 102 is inserted into a loop 304 disposed inside the strap 102. When the strap 102 is adjusted as per the preferences of the wearer, then, one of the locking pins 306 disposed inside the loop 304 engages with one of the apertures 106 to lock the strap 102. The locking pin is unfastened only when the guardian key is received by the control unit 104, thereby preventing unlocking of the wristband 100 by the wearer. In some embodiments, a preset timer such as 1 hour, 2 hours and more can be preconfigured to automatically (i.e., autonomously) unlock the device 100. The locking mechanism of the device 100 prevents wearers, especially children or individuals with cognitive impairments, from removing the wristband themselves.

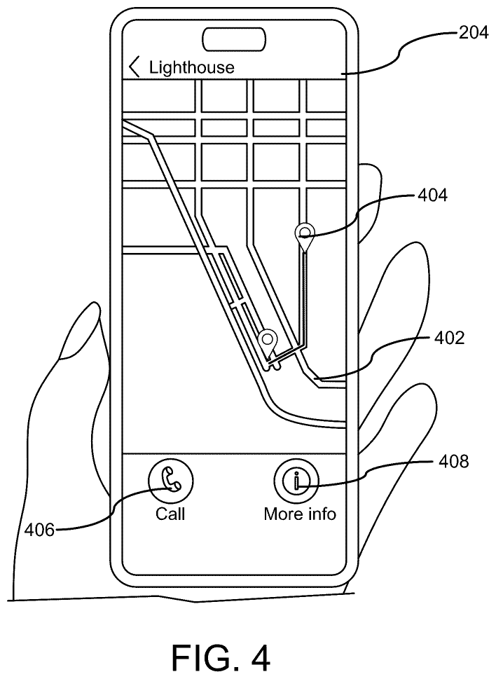

FIG. 4 illustrates an exemplary user interface displayed by the software application for displaying location of the wearer of the safety and tracking device in accordance with the disclosed architecture. The software application 204 displays a digital map 402 which shows the real time location 404 of the wearer of the device 100. The application 204 uses the GPS data collected by the wristband 100 and displays it on the map 402, enabling instant tracking and quick responses to emergencies. In some embodiments, historical data and movement patterns of the wearer may also be visualized on the map 402 for further insights.

A contact icon 406 is provided on the interface 400 which enables a caregiver to directly send a message or text to the device 100 which is displayed on the touch screen 120 of the device 100. A more information icon 408 when selected displays the historical data and geofence data of the device 100.

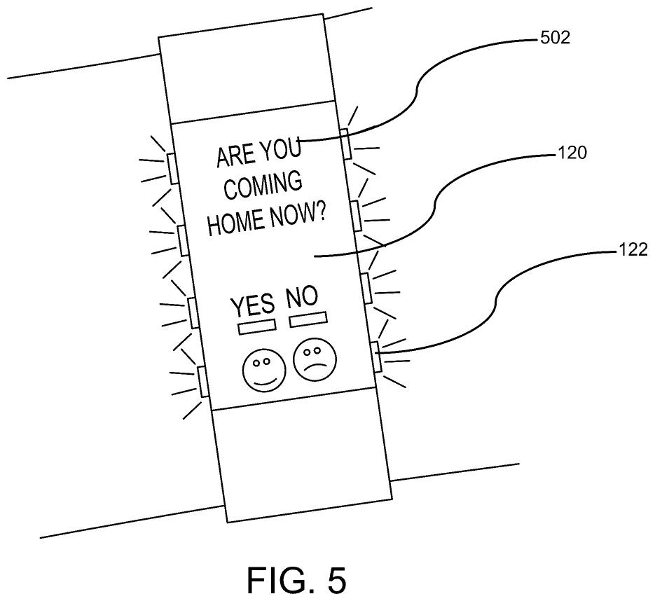

FIG. 5 illustrates a text message being displayed on the touch screen of the safety and tracking device of the present invention in accordance with the disclosed structure. As illustrated, the device 100 can receive a text message 502 from the software application 204 and the message 502 is displayed on the touch screen 120. A response can also be sent using the touch screen 120, thereby eliminating use of any additional device for communication between the wearer of the device 100 and the remote caregiver or parent (i.e., paired remote device). When the message is displayed on the touch screen 120, then, the LEDs 122 are illuminated for providing a visual notification to the wearer of the device 100.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “safety and tracking wearable device”, “wearable safety device”, “wearable location tracking and safety device”, “wristband”, “wearable device”, and “device” are interchangeable and refer to the location tracking and safety wearable device 100 of the present invention.

Notwithstanding the forgoing, the location tracking and safety wearable device 100 of the present invention can be of any suitable size and configuration as is known in the art without affecting the overall concept of the invention, provided that it accomplishes the above stated objectives. One of ordinary skill in the art will appreciate that the location tracking and safety wearable device 100 as shown in the FIGS. are for illustrative purposes only, and that many other sizes and shapes of the location tracking and safety wearable device 100 are well within the scope of the present disclosure. Although the dimensions of the location tracking and safety wearable device 100 are important design parameters for user convenience, the location tracking and safety wearable device 100 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A safety and tracking wearable device comprising:
a smart band;
a control sensor unit;
an alert button;
a wireless module; and
a paired remote device;
wherein said smart band is a wristband;
wherein said control sensor unit having a GPS module for tracking a real time location of a wearer of said smart band;
wherein said GPS module continuously tracks the real time location of the wearer of said smart band;
wherein said wireless module establishes a communication channel with said paired remote device; and
further wherein said alert button upon actuation identifies the real time location of the wearer of said smart band and sends a predetermined alert message to said paired remote device.

2. The safety and tracking wearable device of claim 1 further comprising a vital sensor, wherein said vital sensor detects heart rate.

3. The safety and tracking wearable device of claim 2, wherein said smart band sends a notification to said paired remote device when said vital sensor does not detect any information from the wearer.

4. The safety and tracking wearable device of claim 3, wherein said not detecting any information is for a predetermined period of time.

5. The safety and tracking wearable device of claim 4, wherein said smart band having a touch display for displaying a text message received from said paired remote device.

6. The safety and tracking wearable device of claim 5, wherein said touch display for sending a text message to said paired remote device.

7. The safety and tracking wearable device of claim 6, wherein said smart band having a plurality of LEDs, and further wherein said LEDs illuminate upon sending said predetermined alert message to said paired remote device.

8. The safety and tracking wearable device of claim 7, wherein said LEDs illuminate upon receipt of the text message from said paired remote device.

9. The safety and tracking wearable device of claim 6, wherein said smart band having a proximity range detector, and further wherein said smart band sends a notification to said paired remote device when said proximity range detector is outside of a preconfigured geofence area.

10. The safety and tracking wearable device of claim 9, wherein said paired remote device is a smartphone.

11. A safety and tracking wearable device comprising:
a smart band;
a selectively removable strap;
a control sensor unit;
an alert button;
a wireless module; and
a paired remote device;
wherein said smart band is a wristband;
wherein said control sensor unit having a GPS module for tracking a real time location of a wearer of said smart band;
wherein said GPS module continuously tracks the real time location of the wearer of said smart band;
wherein said wireless module establishes a communication channel with said paired remote device;
wherein said alert button upon actuation identifies the real time location of the wearer of said smart band and sends a predetermined alert message to said paired remote device;
further wherein said selectively removable strap having a digital key for controlled locking and unlocking of said selectively removable strap.

12. The safety and tracking wearable device of claim 11 further comprising a vital sensor, wherein said vital sensor detects heart rate.

13. The safety and tracking wearable device of claim 12, wherein said smart band sends a notification to said paired remote device when said vital sensor does not detect any information from the wearer.

14. The safety and tracking wearable device of claim 13, wherein said not detecting any information is for a predetermined period of time.

15. The safety and tracking wearable device of claim 14, wherein said smart band having a touch display for displaying a text message received from said paired remote device.

16. The safety and tracking wearable device of claim 15, wherein said touch display for sending a text message to said paired remote device.

17. The safety and tracking wearable device of claim 16, wherein said smart band having a proximity range detector, and further wherein said smart band sends a notification to said paired remote device when said proximity range detector is outside of a preconfigured geofence area.

18. The safety and tracking wearable device of claim 9, wherein said paired remote device is a smartphone.

19. A safety and tracking wearable device comprising:
a smart band;
a selectively removable strap;
a control sensor unit;
an alert button;
a wireless module;
an application; and
a paired remote device;
wherein said smart band is a wristband;
wherein said control sensor unit having a GPS module for tracking a real time location of a wearer of said smart band;
wherein said GPS module continuously tracks the real time location of the wearer of said smart band;
wherein said wireless module establishes a communication channel with said paired remote device;
wherein said alert button upon actuation identifies the real time location of the wearer of said smart band and sends a text message to said paired remote device;
wherein said selectively removable strap having a digital key for controlled locking and unlocking of said selectively removable strap;
wherein said paired remote device having a digital map;
further wherein said application shows the real time location of the wearer of said smart band on said digital map.

20. The safety and tracking wearable device of claim 19, wherein said smart band having a proximity range detector, and further wherein said smart band sends a notification to said paired remote device when said proximity range detector is outside of a preconfigured geofence area.
